(19) United States
(12) Patent Application Publication
(10) Pub. No.: US 2012/0130133 A1
(43) Pub. Date: May 24, 2012

(54) Method For Synthesizing Vitamin D Analogs

(75) Inventor: Uttam Saha, Thornhill (CA)

(73) Assignee: CYTOCHROMA INC., Markham, ON (CA)

(21) Appl. No.: 13/131,176

(22) PCT Filed: Nov. 25, 2009
(86) PCT No.: PCT/CA09/01687

§ 371 Date: Jul. 19, 2011

Related U.S. Application Data
(60) Provisional application No. 61/118,030, filed on Nov. 26, 2008.

(51) Int. Cl. C07C 35/21; C07C 43/03; C07C 43/205

(52) U.S. Cl. 568/648; 568/819; 568/662

(57) ABSTRACT

Processes for preparing vitamin D₂ derivatives and intermediates to vitamin D₂ derivatives are disclosed. An improved photolysis process for the preparation of cis intermediate (I) from the trans starting material (II) are disclosed. Also disclosed is an improved process for the formation of a trans double bond at C₂₂-C₂₃ of the vitamin D₂ derivative, which provides high selectivity of the desired trans double bond of compound (III) over the undesired cis double bond of compound (IIIA).

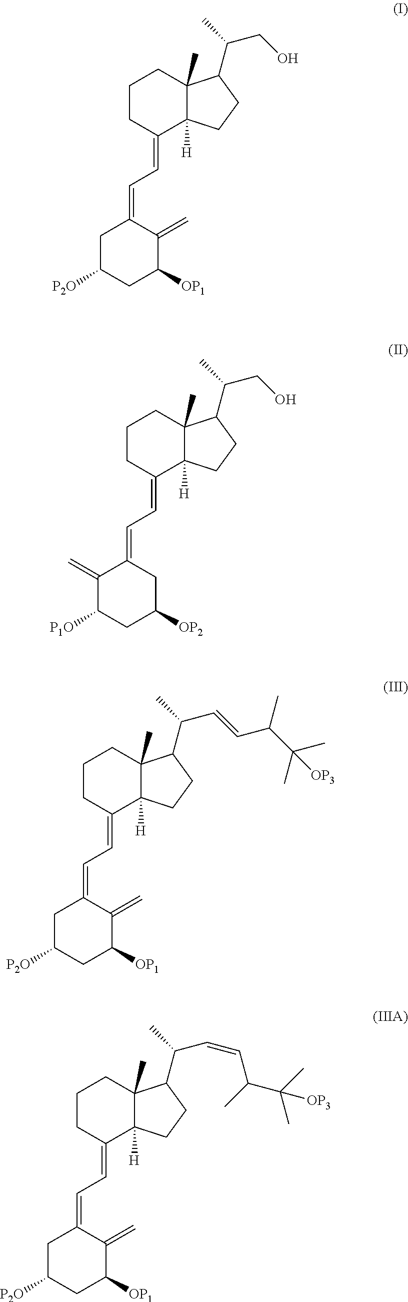

### CROSS REFERENCE TO RELATED APPLICATIONS

The priority benefit under 35 U.S.C. §119(e) of U.S. Provisional Application No. 61/118,030, filed Nov. 26, 2008, is hereby claimed, and the disclosure thereof is incorporated herein by reference in its entirety.

### BACKGROUND

1. Field of the Disclosure

This disclosure relates generally to methods for preparing Vitamin D precursors and analogs. More particularly, this disclosure relates to methods of synthesizing a Vitamin D₂ analog using photolysis and Wittig chemistry.

2. Brief Description of Related Technology

Vitamin D analogs are known to have pharmaceutical activity and are useful for treating various conditions, such as psoriasis and neoplastic disease. Prior known synthetic routes to prepare Vitamin D₂ and analogs thereof have poor selectivity of formation of double bonds and can require multiple purifications to provide product of suitable purity. See, e.g., Coutts, et al., Org. Proc. Res. Dev., 6(3):246-255 (2002) and Kutner, et al., J. Org. Chem., 53:3450-3457 (1988). Thus, a need exists for methods of preparing vitamin D₂ and analogs thereof that provides improved selectivity of double bond formation and greater purity of the final product.

### SUMMARY

Disclosed herein are methods of preparing Vitamin D₂ or analogs thereof, or intermediates for the synthesis of Vitamin D₂ or analogs thereof. Thus, one aspect provides a method of synthesizing a compound of formula (I)

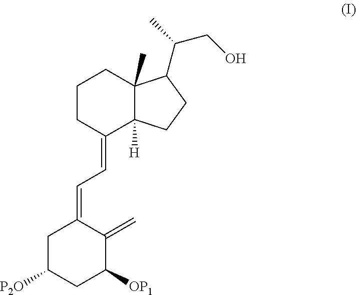

comprising exposing a compound of formula (II) to light form the compound of formula (I),

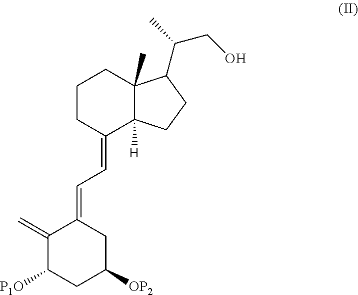

wherein P₁ and P₂ are each independently selected from the group consisting of hydrogen and a hydroxyl protecting group; the light has a wavelength of greater than 360 nm, and the exposing step is performed at a temperature below about 15° C. The wavelength can be greater than 360 nm, and for example can be in a range of 360 nm to 400 nm. P₁ and P₂ can be the same or different. Optionally, the exposing is performed in the presence of 9-acetylanthracene, acridine, phenazine, anthracene, or a combination thereof. Further optionally, the exposing is performed in the presence of an organic base. The organic base can comprise an alkyl amine, for example triethylamine. To provide the exposure light of the desired wavelength, excitation light can be filtered through a uranium filter. The exposing step can be for less than one hour and result in a yield of the compound of formula (I) of greater than 95%. The exposing step can be for less than 45 minutes and result in a yield of the compound of formula (I) of greater than 98%.

Another aspect of the present disclosure provides a method of preparing a compound of formula (III) and optionally a compound of formula (IIIA),

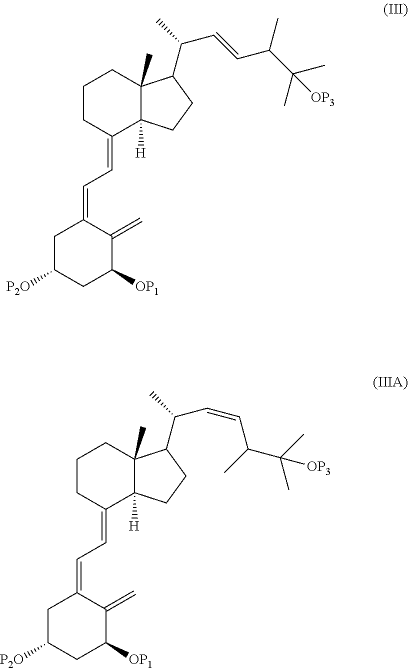

comprising admixing a compound of formula (IV) and a compound of formula (V) to form the compound of formula (III) and optionally the compound of formula (IIIA),

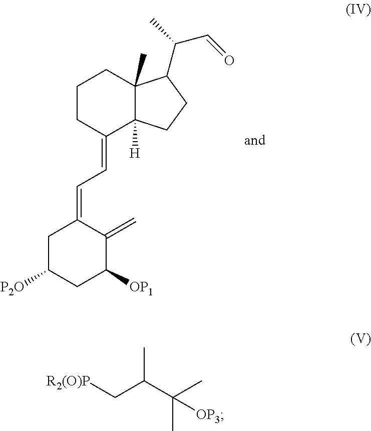

wherein each R is independently an alkyl group or an aryl group; P₁, P₂, and P₃ are each independently selected from the group consisting of hydrogen and a hydroxyl protecting group; and the ratio of the compound of formula (III) to the compound of formula (IIIA) is at least 95:5. R can be methyl, ethyl, propyl, phenyl, substituted phenyl, or naphthyl. P₁ and P₂ can be the same or different. Optionally, at least one of P₁, P₂, and P₃ is a silyl protecting group. The ratio of the compound of formula (III) to the compound of formula (IIIA) preferably is at least 98:2 or at least 99:1. The compound of formula (V) can have a stereochemistry of

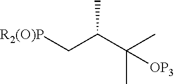

and the compound of formula (III) can have a stereochemistry of

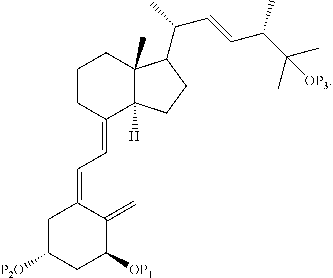

In embodiments where at least one of P₁, P₂, or P₃ is not hydrogen, the method can further comprise removing the non-hydrogen hydroxyl protecting groups of P₁, P₂, and P₃ to form the compound of formula (III) such that each of P₁, P₂, and P₃ is hydrogen. In these embodiments, the method also can further comprise crystallizing the compound of formula (III) from a solvent mixture comprising acetone and water to provide crystals of the compound of formula (III) having at least 99% or at least 99.5% purity by weight in a single crystallization step.

Crystallization of the compound of formula (III) can alternatively comprise crystallizing the compound of formula (III) from t-butyl methyl ether (tBuOMe) to provide crystals of the compound of formula (III) having at least 99%, at least 99.5%, or at least 99.7% purity by weight in a single crystallization step.

Preferably, the crystals are free of methyl formate.

Optionally, the method further comprises drying the crystals under vacuum and at a temperature greater than 35° C., for example about 40° C.

Another aspect of the disclosure provides a compound of formula (V)

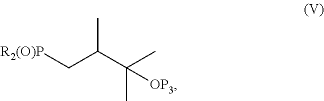

wherein each R is independently an alkyl group or an aryl group and P₃ is hydrogen or a hydroxyl protecting group. P₃ can be a silyl group. R can be methyl, ethyl, propyl, phenyl, substituted phenyl, or naphthyl. Optionally, the compound of formula (V) has a stereochemistry of

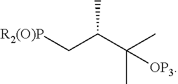

For the compositions and methods described herein, preferred features, such as components, compositional ranges thereof, substituents, conditions, and steps, can be selected from the various examples provided herein.

Further aspects and advantages will be apparent to those of ordinary skill in the art from a review of the following detailed description. While the method is susceptible of embodiments in various forms, the description hereafter includes specific embodiments with the understanding that the disclosure is illustrative, and is not intended to limit the invention to the specific embodiments described herein.

### DETAILED DESCRIPTION

Disclosed herein are improved processes for the preparation a compound of formula (I) and a compound of formula (III). One improved processes involves a photolysis reaction which has a faster reaction time and provides a greater conversion to the desired cis compound (I), than prior methods. Another improved process involves formation of a compound of formula (III) which provides greater selectively of the trans double bond at C22, C23 and less formation of the undesired cis compound of formula (IIIA), than prior methods. The resulting compound of formula (III) can then be deprotected and the resulting vitamin D₂ compound can be purified in fewer crystallizations.

A compound of formula (I) can be formed by exposing a compound of formula (II) to light

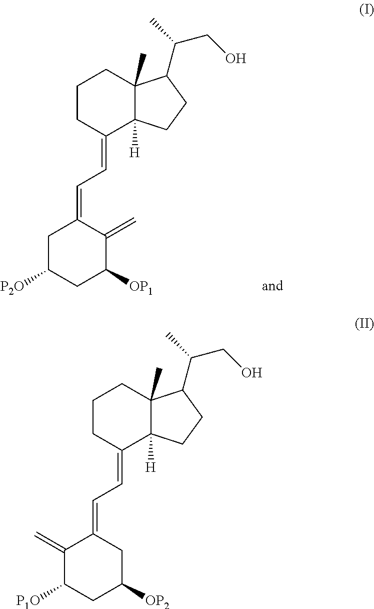

wherein P₁ and P₂ are each independently selected from the group consisting of hydrogen and a hydroxyl protecting group. The light preferably has a wavelength greater than 360 nm. For example, the light can have a wavelength in a range of 360 nm to 400 nm. Light having such wavelengths can be obtained, for instance, by filtering the light from an ultraviolet lamp through a uranium filter. Other means for obtaining light having the recited wavelength include use of chemical solutions such as dichromate and the like.

The compound of formula (II) preferably is exposed to the appropriate wavelength of light at a reduced temperature, for example about 15° C. or below. The temperature can be about 10° C. or below, about 5° C. or below, or about 0° C. or below. Other contemplated temperature values fall within the ranges of about −10° C. to about 15° C., about −7° C. to about 10° C., and about −5° C. to about 7° C.

The amount of time that the compound of formula (II) needs to be exposed to light to form the compound of formula (I) in high yield can be much shorter than the time required in prior methods. Prior methods required exposure to light of greater than 400 minutes, and conversion of the starting material still had not gone to completion (see, for example, Shimizu, et al., Chem. Pharm. Bull. 49(3) 312-317 (2001)). In the method disclosed herein, the compound of formula (I) can be formed in greater than 95% yield in less than one hour exposure to light. Preferably, the compound of formula (I) can be formed in greater than 98% yield in less than 45 minutes exposure to light. The conversion of the compound of formula (II) to formula (I) can be monitored by, e.g., HPLC, by analyzing aliquots of the reaction mixture at various times. Therefore, the time of exposing the compound of formula (II) to light can be much shorter than 45 minutes, and the conversion can be greater than 98%, as determined by an analytical technique, such as HPLC.

The mixture that is exposed to light can further include an organic base. The organic base can be any organic base that is compatible with the reaction conditions, but is preferably an alkyl amine. The base is used to prevent, minimize, or avoid the cleavage of a protecting group on the compound, especially the cleavage of a silyl protecting group. Alkyl amines can be monoalkyl amines, dialkylamines, or trialkylamines. The alkyl group(s) on the amine can be the same or different. Typically, the alkyl group will have one to ten carbons, branched, unbranched, or cyclic. Examples of alkyl groups include, but are not limited to, methyl, ethyl, propyl, isopropyl, butyl, isobutyl, sec-butyl, and pentyl, hexyl, cyclohexyl, heptyl, octyl, nonyl, and decyl. A preferred alkyl amine is triethylamine.

Also disclosed herein is a method for preparing a compound of formula (III), and optionally a compound of formula (IIIA), from a compound of formula (IV) and a compound of formula (V)

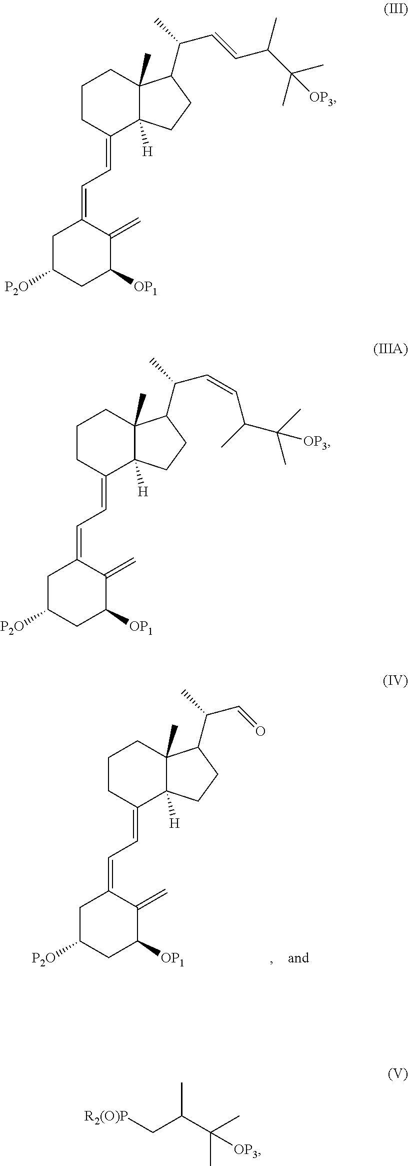

wherein each R is independently an alkyl group or an aryl group, P₁, P₂, and P₃ are each independently selected from the group consisting of hydrogen and a hydroxyl protecting group, and the ratio of the compound of formula (III) to the compound of formula (MA) is at least 95:5. The compound of formula (III) is prepared by reacting the aldehyde of the compound of formula (IV) and the phosphine oxide of the compound of formula (V) in a Wittig reaction. The selectivity of the formation of compound of formula (III) compared to the compound of formula (IIIA) is at least 95:5. The selectivity can be at least 98:2, or at least 99:1.

The R alkyl group or aryl group of the compound of formula (V) can be any alkyl group or aryl group compatible with the Wittig reaction. Preferably, R is a methyl, ethyl, propyl, phenyl, substituted phenyl, or naphthyl. The stereochemistry of the compound of formula (V) can be either (R) or (S), or a mixture of (R) and (S). Optionally, the compound of formula (V) can have the stereochemistry of

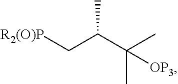

and the compound of formula (III) can have the stereochemistry of

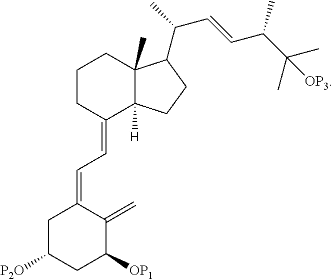

In any of the above processes of the invention, P₁, P₂, and P₃ can be any appropriate hydroxyl protecting group. The choice of an appropriate protecting group is within the skill of the artisan. For example, suitable protecting groups are described in Wuts et al., Greene's Protective Groups in Organic Synthesis, 4th ed., (Wiley Interscience: Hoboken, N.J.) 2007. By hydroxylprotecting group is meant any compound for protecting a hydroxyl group during a chemical reaction (preferably such that the hydroxyl group is easily reinstated), specifically during acidic or basic hydrolysis. A silyl protecting group, such as tert-butyl dimethyl silyl (“TBDMS” or “TBS”) or triethyl silyl (“TES”), is preferred.

The compound of formula (III) can be deprotected to remove any non-hydrogen P₁, P₂, and P₃ to provide a vitamin D₂ derivative compound. Deprotection of hydroxyl protecting groups is within the knowledge of the skilled artisan, and guidance can be found in Wuts et al., Greene's Protective Groups in Organic Synthesis, 4th ed., (Wiley Interscience: Hoboken, N.J.) 2007. For example, when a hydroxyl protecting group is a silyl ether, the silyl ether can be removed by exposure to acidic conditions or to a fluoride source, such as tetrabutylammonium fluoride.

The deprotected compound of formula (III), i.e., wherein each of P₁, P₂, and P₃ is hydrogen, can be crystallized to provide crystals of the compound of formula (III) having a purity of at least 99% by weight in a single crystallization step. Prior crystallization methods of the compound of formula (III) have used the solvent methyl formate (see U.S. Pat. No. 6,903,083). Without intending to be bound by any particular theory, it is believed that methyl formate may be de-stabilizing to the crystals and/or the compound of formula (III). Accordingly, crystallization methods that are free of methyl formate are preferred. Crystallization of the compound of formula (III) is performed by dissolving the crude compound of formula (III) in a solvent, such as either (1) an acetone/water mixture or (2) t-butyl methyl ether. The ratio of acetone to water (by volume) can be in a range of about 5:1 to about 1:5. Specific ratios include, but are not limited to, 5:1, 4:1, 3:1, 2:1, 1:1, 1:2, 1:3, 1:4, and 1:5.

Because the methods disclosed herein provide higher selectivity of formation of desired products (e.g., compound of formula (III) over compound of formula (IIIA)), the resulting crude compound of formula (III) has higher purity than prior methods. Thus, a single crystallization can be sufficient to provide the compound of formula (III) at the desired purity level. Crystals after a single crystallization can be at least 99% pure by weight, at least 99.5% pure by weight, or at least 99.7% pure by weight. Crystals can then be dried to remove any residual solvent under vacuum at elevated temperatures (e.g., above 30° C., or in a range of about 35° C. to about 45° C.) or at ambient temperatures (e.g., about 20° C. to about 25° C.), then stored under an inert gas (e.g., nitrogen or argon) at temperatures below 10° C., below 0° C., or below −10° C.

### EXAMPLES

The following examples are provided for illustration and are not intended to limit the scope of the invention.

### Example 1

### Preparation of Cis-Alcohol Intermediate 2

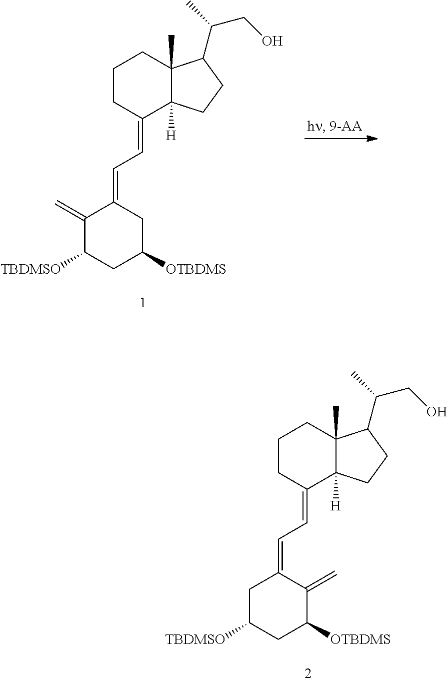

Starting material trans-alcohol 1 (6 g; 10.434 mmol) was placed in a flask with 9-acetylanthracene (0.597 g; 2.710 mmol) and freshly distilled triethylamine (0.015 mL; 0.103 mmol) with 300 mL of toluene. The mixture was cooled to between about −1.7° C. and 6° C. and stirred under argon. The mixture was then exposed to light from a UV lamp inserted into a uranium filter glass tube. Aliquots of 100 μL were collected at time intervals of 30 min, 45 min, and 60 min, and analyzed for completion via HPLC. The results, shown below in Table 1, indicate that the reaction was complete within 30 minutes.

TABLE 1

| Time Point | % product (2) | % starting material (1) |
|---|---|---|
| 30 min | 98.83 | 1.16 |
| 45 min | 98.75 | 1.25 |
| 60 min | 98.69 | 1.3 |

The reaction mixture then was transferred to a flask and evaporated at 35° C. under vacuum. The residue was dissolved in methylene chloride (CH₂Cl₂), loaded into a silica-gel cartridge and eluted with 0-15% diethyl ether (Et₂O) in CH₂Cl₂. The fractions containing the purified product 2 were then concentrated to dryness, providing a quantitative yield of 2.

### Example 2

### Preparation of Aldehyde 3

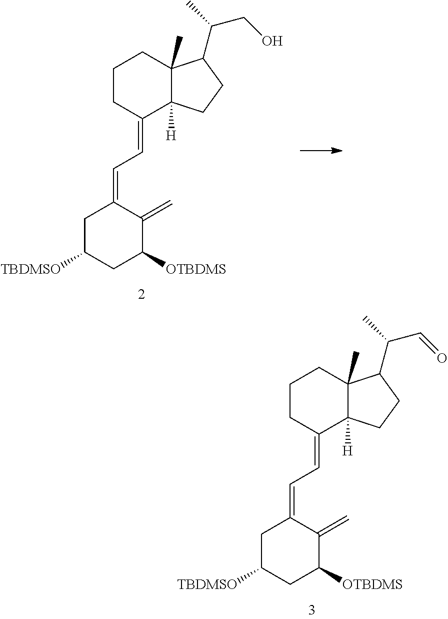

The cis alcohol 2 was oxidized to the aldehyde using sulfur trioxide pyridine complex, following literature procedures (see Tojo and Fernandez, Oxidation of Alcohols to Aldehydes and Ketones, Springer (2006)). Cis-alcohol 2 (6.8 g, 11.82 mmol) was reacted with SO₃.Py (9.4 g; 59.12 mmol) in the presence of 10 mL of freshly distilled triethyl amine, 34 mL CH₂Cl₂, and 68 mL dimethyl sulfoxide (DMSO). The reaction provided quantitative yield of aldehdye 3.

### Example 3

### Preparation of Trans Vitamin D₂ Intermediate 5

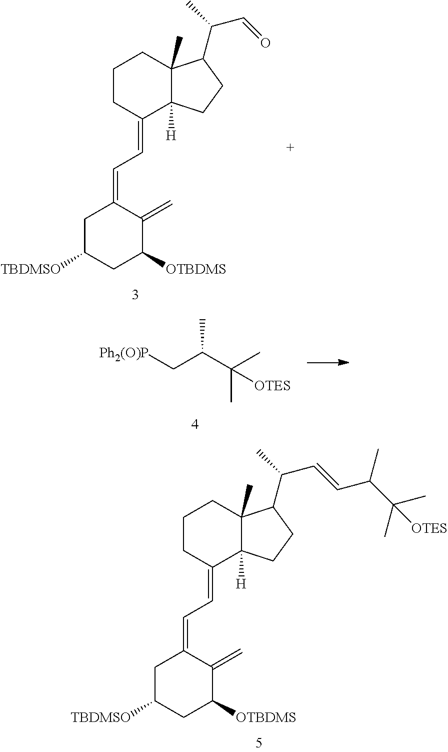

Phosphine oxide 4 (5.8 g, 13.92 mmol) in 75 mL dry tetrahydrofuran (THF) was cooled in a dry ice/acetone bath to about −78° C. under argon. After 10 minutes of cooling, butyl lithium (11.14 mL, 27.84 mmol, 2.5 M in hexanes) was added slowly by syringe. The resulting mixture was stirred for 45 minutes at about −78° C. Aldehyde 3 dissolved in 40 mL anhydrous THF then was added to this mixture via syringe. This resulting mixture was stirred for 45 minutes at −78° C., then allowed to warm to about 0° C. over 45 minutes to 1.5 hours. Then, the reaction was stopped and 200 mL of ethyl acetate was added to the mixture, which was then washed with brine and water. The organic layer was dried over sodium sulfate, filtered and concentrated. The thick syrup concentrate was dissolved in 200 mL anhydrous THF and cooled in an ice salt bath to about −12° C. To this cooled solution was added potassium t-butoxide (1.98 g, 17.74 mmol) and the resulting mixture stirred for 2.5 hours at about −12° C. Another equivalent of the potassium t-butoxide was added and the mixture stirred for an additional hour. The reaction was stopped, and 200 mL of ethyl acetate was added. The mixture was washed with 0.01 N HCl and brine. The organic layer was dried with sodium sulfate and concentrated. The crude mixture was purified with column chromatography (1% ethyl acetate in hexane and 0.01% triethylamine) to give 4.2 g (46% yield) of the intermediate 5. Characterization by ¹H NMR did not show formation of any of the undesired cis olefin at C22-C23.

### Example 4

### Formation of 1,25-dihydroxy Vitamin D₂ Compound 6

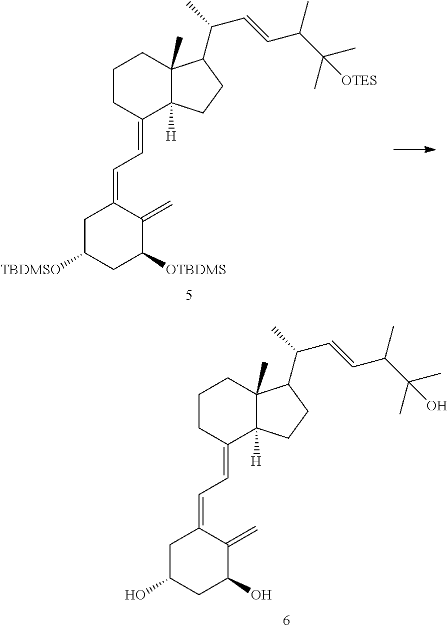

Intermediate 5 (4.2 g) was dissolved in anhydrous THF, and 55 mL tetrabutyl ammonium fluoride was added. The reaction was heated to 50-55° C., monitored by thin layer chromatography for completion. The crude material was purified by column chromatography to provide 1.8 g (77% yield) of the 1,25-dihydroxy vitamin D₂ compound 6.

### Example 5

### Purification

1,25-dihydroxy vitamin D₂ compound 6 obtained from Example 4 was treated with maleic anhydride (40 mg) in THF at room temperature. The reaction was monitored by HPLC. After completion, the solution was evaporated and purified by column chromatography to provide 1,25-dihydroxy vitamin D₂ 6 (1.76, 98% yield). The purity was analyzed using HPLC and found to be 97.89% pure.

### Example 6

### Crystallization using Acetone/Water

The resulting 97.89% pure 1,25-dihydroxy vitamin D₂ compound 6 was then crystallized with an acetone/water mixture as follows. The 1,25-dihydroxy vitamin D₂ compound 6 was first refluxed with acetone (15 ml/1 g) until a clear solution was obtained. It was then filtered and an equal volume of water was gradually added. Once the temperature reached about 25° C., crystal formation started and the flask was placed at 4° C. freezer for 24 h. The solid was filtered and washed with pre-chilled 1:1 acetone/water at 4° C. After this single crystallization, the purity, measured by HPLC, of the resulting 1,25-dihydroxy vitamin D₂ compound 6 was 99.8%.

### Example 7

### Crystallization using t-Butyl Methyl Ether

1,25-Dihydroxy vitamin D₂ compound 6 (13.3 g, pre-vitamin >2.0%) was taken in a three neck flask equipped with a magnetic stir bar and N₂ inlet/outlet. A reflux condenser and an addition funnel were attached. t-Butyl methyl ether (665 mL) was charged to the flask, and the resulting solution was refluxed and stirred vigorously. A clear solution was obtained after 27 minutes. The heating was ceased, and, while the solution was still vigorously stirred, heptane (1330 mL) was added to the solution using a dropping funnel, at a rate of about 200 ml/min. Once addition was complete, the solution was removed from the heating mantle, and covered with aluminum foil to cool to ambient temperature (cooling time about 7.5 hours). The solution was then placed in a −20° C. freezer over night (about 15 hours). The resulting crystals were then filtered through a sintered glass funnel and washed twice (200 mL each) with a pre-cooled tBuOMe/heptane solvent mixture (1:2 by volume). The crystals were then grinded to powder and dried under vacuum at ambient temperature for 48 hours. After this single crystallization, the purity, measured by HPLC, of the resulting 1,25-dihydroxy vitamin D₂ compound 6 was 99.7% with pre-vitamin content of about 0.05%.

The foregoing description is given for clearness of understanding only, and no unnecessary limitations should be understood therefrom, as modifications within the scope of the invention may be apparent to those having ordinary skill in the art.

Throughout the specification, where methods are described as including steps, components, or materials, it is contemplated that the compositions can also consist essentially of, or consist of, any combination of the recited steps, components or materials, unless described otherwise.

The practice of a method disclosed herein, and individual steps thereof, can be performed manually and/or with the aid of electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A method of synthesizing a compound of formula (I) 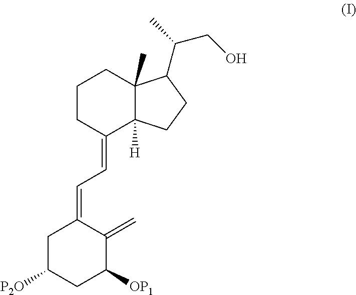
comprising exposing a compound of formula (II) to light form the compound of formula (I), 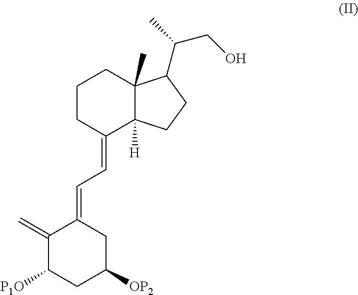
wherein P₁ and P₂ are each independently selected from the group consisting of hydrogen and a hydroxyl protecting group; the light has a wavelength of greater than 360 nm, and the exposing step is performed at a temperature below about 15° C.

2. The method of claim 1, wherein the light has a wavelength of greater than 360 nm to 400 nm.

3. The method of claim 1, wherein the exposing of the compound of formula (II) to light is performed in the presence of 9-acetylanthracene, acridine, phenazine, anthracene, or a combination thereof.

4. The method of claim 1, wherein the exposing of the compound of formula (II) to light is performed in the presence of an organic base.

5. The method of claim 4, wherein the organic base comprises an alkyl amine.

6. The method of claim 5, wherein the alkyl amine comprises triethylamine.

7. The method of claim 1, wherein at least one of P₁ and P₂ is a silyl protecting group.

8. The method of claim 1, wherein P₁ and P₂ are the same.

9. The method of claim 1, wherein the light is filtered through a uranium filter.

10. The method of claim 1, wherein the compound of formula (II) is exposed to light for less than one hour and the compound of formula (I) is formed in greater than 95% yield.

11. The method of claim 10, wherein the compound of formula (II) is exposed to light for less than 45 minutes and the compound of formula (I) is formed in greater than 98% yield.

12. A method for preparing a compound of formula (III) 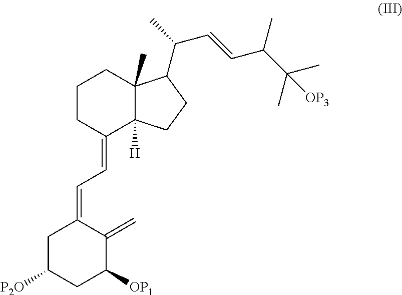
and optionally a compound of formula (IIIA) 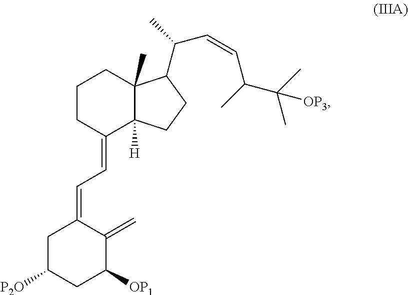
comprising admixing a compound of formula (IV) and a compound of formula (V) to form the compound of formula (III) and optionally the compound of formula (IIIA), 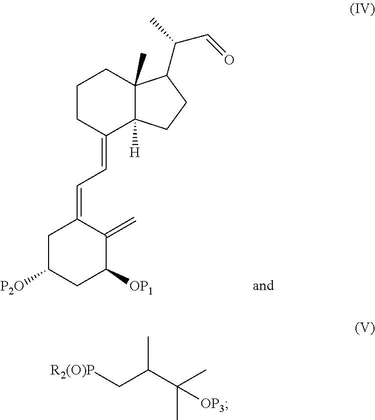
wherein each R is independently an alkyl group or an aryl group, P₁, P₂, and P₃ are each independently selected from the group consisting of hydrogen and a hydroxyl protecting group, and the ratio of the compound of formula (III) to the compound of formula (IIIA) is at least 95:5.

13. The method of claim 12, wherein at least one R is phenyl.

14. The method of claim 12, wherein P₁ and P₂ are the same.

15. The method of claim 12, wherein at least one of P₁, P₂, and P₃ is a silyl protecting group.

16. The method of claim 12, wherein the ratio of the compound of formula (III) to the compound of formula (IIIA) is at least 98:2.

17. The method of claim 16, wherein the ratio of the compound of formula (III) to the compound of formula (IIIA) is at least 99:1.

18. The method of claim 12, wherein the compound of formula (V) has a stereochemistry of

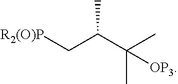

19. The method of claim 18, wherein the compound of formula (III) has a stereochemistry of

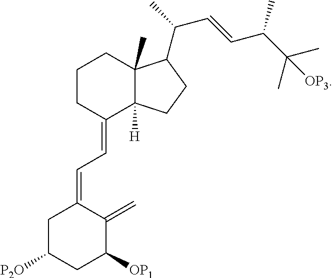

20. The method of claim 12, when at least one of P₁, P₂, or P₃ is not hydrogen, further comprising removing the non-hydrogen hydroxyl protecting groups of P₁, P₂, and P₃ to form the compound of formula (III) such that each of P₁, P₂, and P₃ is hydrogen.

21. The method of claim 20, further comprising crystallizing the compound of formula (III) from a solvent mixture comprising acetone and water to provide crystals of the compound of formula (III) having at least 99% purity by weight in a single crystallization step.

22. The method of claim 20, further comprising crystallizing the compound of formula (III) from t-butyl methyl ether to provide crystals of the compound of formula (III) having at least 99% purity by weight in a single crystallization step.

23. The method of claim 21, wherein the crystals of the compound of formula (III) have a purity of at least 99.5% by weight.

24. The method of claim 21, wherein the crystals of the compound of formula (III) are free of methyl formate.

25. The method of claim 21, further comprising drying the crystals of the compound of formula (III) under vacuum and at a temperature greater than 35° C.

26. The method of claim 25, wherein the temperature is about 40° C.

27. The method of claim 22, further comprising drying the crystals of the compound of formula (III) under vacuum at ambient temperature.

28. A compound of formula (V) 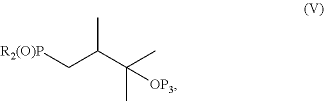
wherein each R is independently an alkyl group or an aryl group and P₃ is hydrogen or a hydroxyl protecting group.

29. The compound of claim 28, wherein P₃ is a silyl group.

30. The compound of claim 28, wherein P₃ is hydrogen.

31. The compound of claim 28, wherein at least one R is phenyl.

32. The compound of claim 28 having the stereochemistry

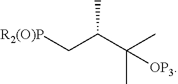

33. The method of claim 22, wherein the crystals of the compound of formula (III) have a purity of at least 99.5% by weight.

34. The method of claim 22, wherein the crystals of the compound of formula (III) are free of methyl formate.
